(19) United States
(12) Patent Application Publication
(10) Pub. No.: US 2018/0256275 A1
(43) Pub. Date: Sep. 13, 2018

(54) CORNER DRAWER FOR ACCESSORY CABINET

(71) Applicant: Biotec Inc., Zeeland, MI (US)

(72) Inventors: Ross A. DeJong, Hudsonville, MI (US); Nate W. Petroelje, West Olive, MI (US)

(73) Assignee: Biotec Incorporated

(21) Appl. No.: 15/913,986

(22) Filed: Mar. 7, 2018

Related U.S. Application Data
(60) Provisional application No. 62/468,155, filed on Mar. 7, 2017.

(51) Int. Cl. A61B 50/18; A47B 88/40

(52) U.S. Cl. CPC A61B 50/18; A61B 2050/105; A47B 2210/0056; A47B 88/402

(57) ABSTRACT

A corner support or drawer for a medical or dental cabinet includes a support panel that is configured to be generally horizontally arranged relative to the cabinet. A face panel of the corner drawer may be attached at the bottom panel to border at least two sides of the storage area. The face panel includes a front portion configured to generally align with a front face of the cabinet and a side portion configured to generally align with a side surface of the cabinet. The support or drawer is configured to travel, generally horizontally, along a linear path that is not parallel with the front face of the cabinet and is not parallel with the side surface of the cabinet.

### CROSS REFERENCE TO RELATED APPLICATION

The present application claims the filing benefits of U.S. provisional application Ser. No. 62/468,155, filed Mar. 7, 2017, which is hereby incorporated herein by reference in its entirety.

### FIELD OF THE INVENTION

The present invention generally relates to a drawer configuration for a cabinet or console, and more particularly to a drawer and cabinet for use in a medical or dental operatory.

### BACKGROUND OF THE INVENTION

It is generally known to provide cabinets or consoles in a dental operatory to provide storage and counter space for use by a dental assistant or dentist or orthodontist or other dental or medical personnel. It is also appreciated that these dental cabinets and consoles may have sinks, power receptacles, dental device hook-ups, waste receptacles, drawers, and cabinet doors, among other features that are arranged to generally accommodate such a dental operatory setting.

### SUMMARY OF THE PRESENT INVENTION

The present invention provides a cabinet for a dental operatory that includes a corner drawer arranged to extend or open from the cabinet at an angle and direction that provides a desirable and unique location of the drawer in a final open position, such as to conveniently provide tools and/or materials stored or held in the drawer to a dental assistant or dentist or the like. The corner drawer includes an exterior portion, such as a face panel, that generally borders at least two sides of the drawer storage area, such that a front portion of the face panel may substantially align with a front surface of the dental cabinet and a side portion of the face panel may substantially align with a side surface of the dental cabinet. The corner drawer is arranged to travel, such as via a slider or moving mechanism or drawer slide (comprising a track/rail configuration with rollers or bearings that roll to facilitate smooth movement of the drawer relative to the cabinet), along a linear path that is at an angle that is not parallel with the front surface or the side surface of the dental cabinet, such as at an angle of about 20 degrees to about 70 degrees, or about thirty-five degrees to about fifty-five degrees, or about forty degrees to about fifty degrees from either surface (such as, for example, about forty-five degrees from either surface). The direction of travel is designed so that the final open position of the corner drawer is such that the contents of the drawer are easily accessible to the assistant or dentist, considering the location(s) where they perform their dental procedures. For instance, this cabinet could, optionally, be located on either side of a dental operatory, such that the drawer may open in an angled direction toward the head of the patient.

According to one aspect of the present invention, a cabinet for a dental operatory includes a base unit that has a side wall and a front wall that are substantially perpendicular relative to each other. A drawer of the cabinet has a face panel that at least partially borders the storage area of the drawer. Such storage area may be generally defined by a plurality of vertical containment walls or panels that each attach at a bottom panel to define the drawer structure. The face panel may also include a front portion that is substantially aligned with the front wall of the base unit and a side portion that is substantially aligned with the side wall of the base unit. The drawer is slidably or movably disposed or attached at the base unit to travel generally horizontally at an angle from the base unit, such as an oblique angle, that is not parallel with the front wall or the side wall of the base unit.

According to another aspect of the present invention, a corner drawer is provided for a dental cabinet. The corner drawer includes a bottom panel that generally defines a drawer storage area and is configured to be generally horizontally arranged relative to the associated dental cabinet. A face panel of the corner drawer is attached at the bottom panel to border at least two sides of the drawer storage area. The face panel includes a front portion configured to substantially align with a front face of the dental cabinet and a side portion configured to substantially align with a side surface of the dental cabinet. The corner drawer is configured to travel at an angle that is not parallel with the front face or the side surface of the dental cabinet.

These and other objects, advantages, purposes, and features of the present invention will become apparent upon review of the following specification in conjunction with the drawings.

### BRIEF DESCRIPTION OF THE DRAWINGS

FIG. 1 is an upper perspective view of a dental accessory cabinet, in accordance with the present invention;

FIG. 2 is an upper perspective view of the dental accessory cabinet shown in FIG. 1, illustrating a corner drawer extended to an open position;

FIG. 3 is an upper perspective view of the dental accessory cabinet shown in FIG. 2, illustrating a direction of travel or movement of the corner drawer between the open position and a closed position;

FIG. 4 is an enlarged upper perspective view of a section of the dental accessory cabinet shown in FIG. 2, illustrating the corner drawer in the open position;

FIG. 5 is an upper perspective view of the dental accessory cabinet shown in FIG. 1, taken from an opposing end of the dental accessory cabinet;

FIG. 6 is an upper perspective view of the dental accessory cabinet shown in FIG. 5, illustrating the corner drawer extended to the open position;

FIG. 7 is an upper perspective view of the dental accessory cabinet shown in FIG. 5, illustrating a direction of travel or movement of the corner drawer between the open position and the closed position;

FIG. 8 is a top plan view of the dental accessory cabinet shown in FIG. 1;

FIG. 9 is a top plan view of the dental accessory cabinet shown in FIG. 2;

FIG. 10 is a top plan view of the dental accessory cabinet shown in FIG. 3;

FIG. 11 is a top plan view of the section of the dental accessory shown in FIG. 4; and

FIG. 12 is a top plan view of another dental accessory cabinet with a corner drawer and a central drawer in extended positions, illustrating otherwise hidden features in dashed lines.

### DETAILED DESCRIPTION OF EMBODIMENTS

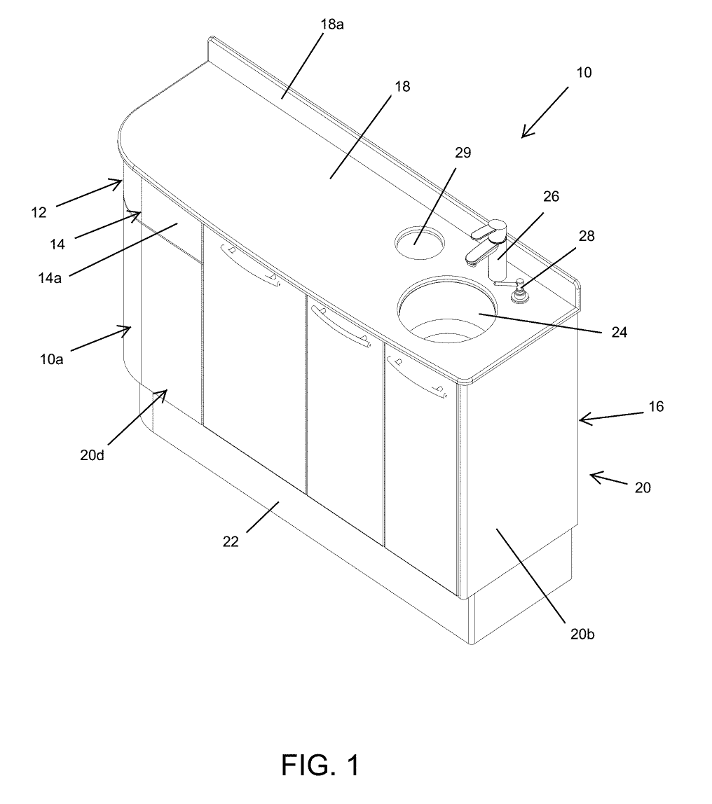

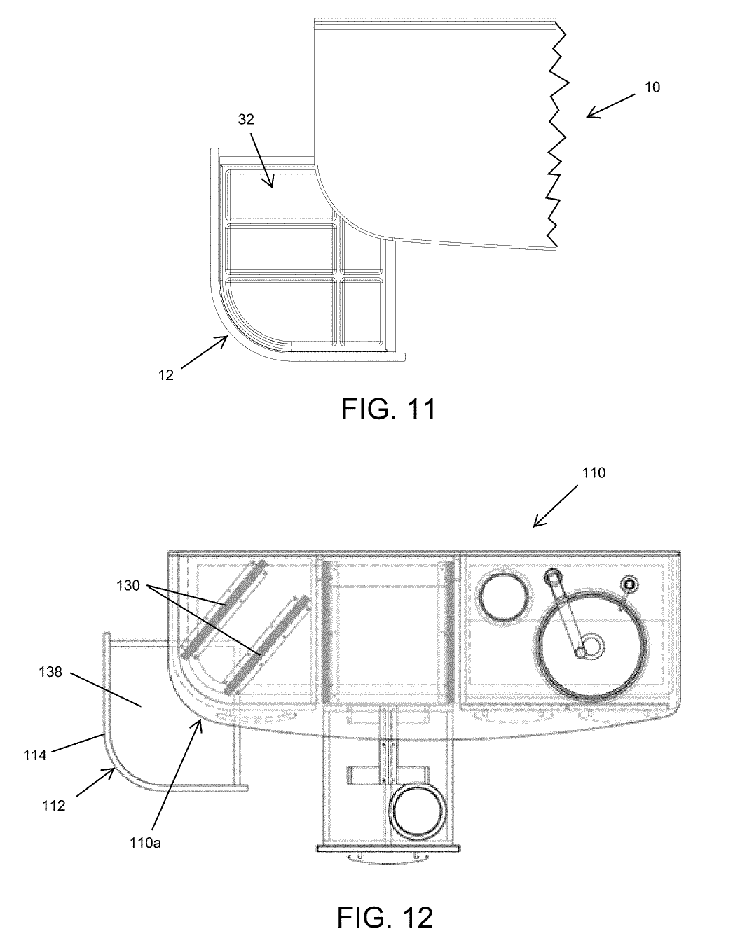

Referring now to the drawings and the illustrative embodiments depicted therein, a medical or dental accessory cabinet 10, 110 (FIGS. 1-11 and FIG. 12) is provided for a medical or dental operatory or operating room, such as to be located on either side of a medical or dental operatory. The cabinet 10, 110 includes a corner drawer 12, 112 that is arranged at a corner portion 10a, 110a of the cabinet, so as to extend or open from the corner portion of the cabinet at an angle and direction that locates the drawer to provide convenient access to tools and/or materials contained in the drawer, such as near the head of the patient for access by a medical or dental assistant or dentist or the like. It is also contemplated that the angled movement of the drawer may position the drawer at a location that reduces interference with an area in front of the cabinet. The corner drawer 12, 112 extends from the cabinet at a generally oblique angle relative to at least some of the walls or vertical surfaces of the cabinet, such that the direction of travel of the drawer is not perpendicular or parallel with the front or side of cabinet. The corner drawer 12, 112 may include an exterior facing portion or surface, such as defined by a face panel 14, 114, which may generally align with one or more of the surrounding exterior surfaces of the corner portion of the cabinet, such as an adjacent section of the side surface of the cabinet and an adjacent section of the front surface of the cabinet.

The cabinet 10, as illustrated in FIGS. 1-3, includes a base structure or unit 16 that may be configured to be mounted or positioned against a wall, such as a building wall or partition wall of a medical or dental operatory (or may be a free standing unit positioned away from a wall with a finished back/sides). The base structure or unit 16 of the cabinet 10 supports a countertop 18 that may include a backsplash, such as a backsplash 18a that integrally extend upward along a rear edge of the countertop 18, as shown in FIGS. 1-4. The illustrated countertop 18 also provides openings for a sink 24, a faucet 26, a soap dispenser 28, and a waste receptacle 29 that are located at an opposing end section of the cabinet 10 from the corner drawer 12. Optional embodiments may provide more or fewer features or accessories at the countertop and they may be provided in various configurators and layouts.

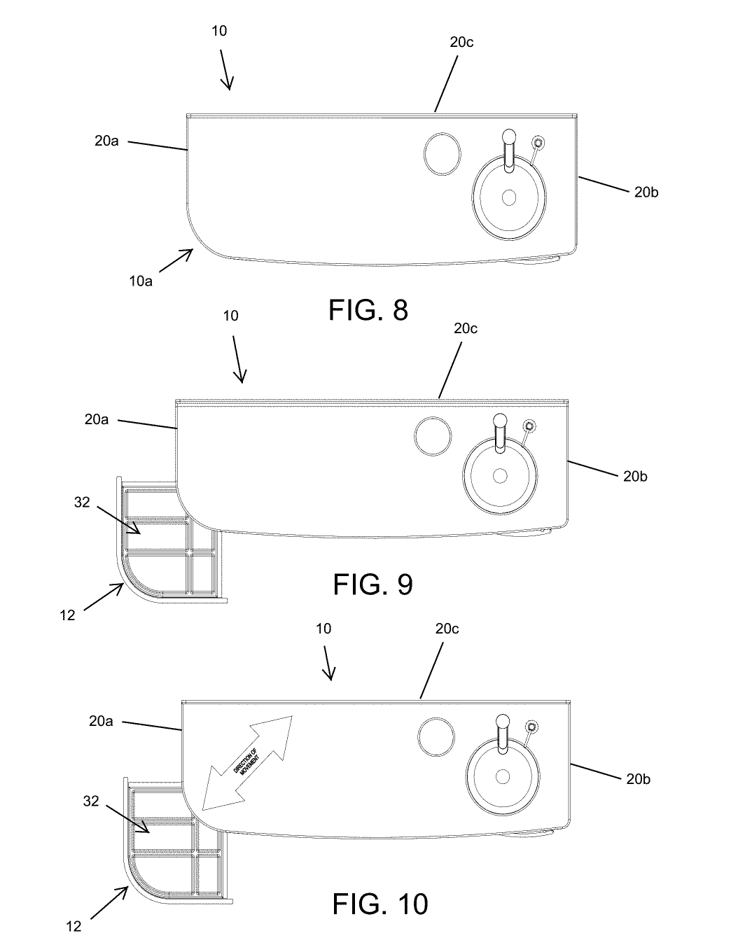

The base structure or unit 16 also includes a cabinet frame 20 that may be supported by a stand or base rail or base assembly 22, such as to provide a front inset toe or kick space. However, it is contemplated that the cabinet may optionally be supported by other stands, such as by feet or casters or the like. The cabinet frame 20 provides a structure for supporting the cabinet storage drawers or shelves or the like. The cabinet frame 20 includes parallel and opposing side walls 20a, 20b, along with a rear wall 20c (FIGS. 8-10) that is generally perpendicular to the side walls 20a, 20b. The cabinet frame 20 also includes a front wall 20d that provides a front or face surface, such as provided by front panels of cabinet doors and/or drawers. The cabinet frame 20 may include a face frame at or near the front wall 20d that provides front openings arranged to receive the front panels of the cabinet doors and/or drawers in a manner that enclose the front openings when such doors or drawers are in a closed position. The front wall 20d is generally opposing and in parallel alignment with the rear wall 20c; however, optionally, the front wall may have a curvature or other shape and may or may not be parallel with the rear wall. The base structure or unit 16 may accommodate other cabinetry, cabinet drawer and door configurations, storage areas, or features that may be useful in a medical or dental operatory.

The corner drawer 12, as shown in FIGS. 1-7, is arranged at the corner portion 10a of the cabinet 10, immediately below the countertop 18 and above a lower curved exterior panel. The lower curved exterior panel may be a stationary cover or form a front to one or more corner doors or additional corner drawers, such as three or four additional corner drawers that slide or move or roll out at generally the same angle as the corner drawer 12 shown and described herein. Optionally, the corner drawer may be provided at a lower location on the cabinet, such that an opening or other storage compartment may be provided between the corner drawer and the countertop.

The corner drawer may comprise a drawer with the face panel and at least one interior or rear panel (that is interior the cabinet when the corner drawer is closed) attached to a bottom panel or support panel to provide a drawer storage area. Optionally, the corner drawer or corner support panel may comprise a shelf panel or support panel without the interior/rear and face panels (having side/front/rear/interior edges about its periphery), and optionally with lower or reduced profile interior/rear and face panels or lips that generally circumscribe the support panel, but not to a level that closes the opening at the cabinet when the support panel is in its closed or retracted state.

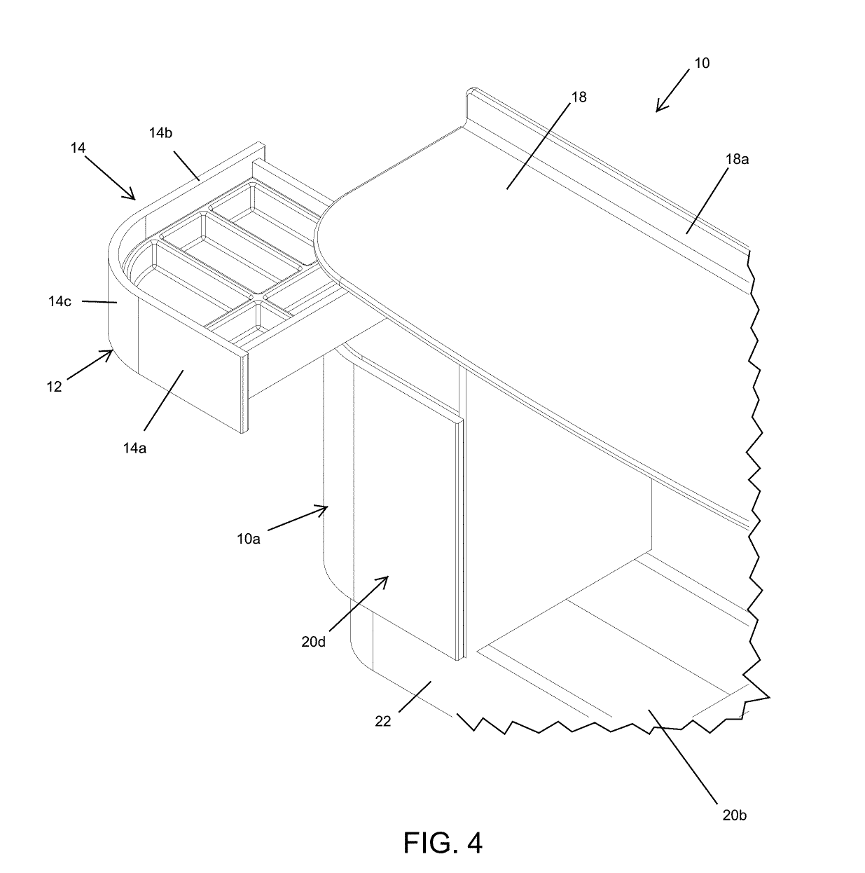

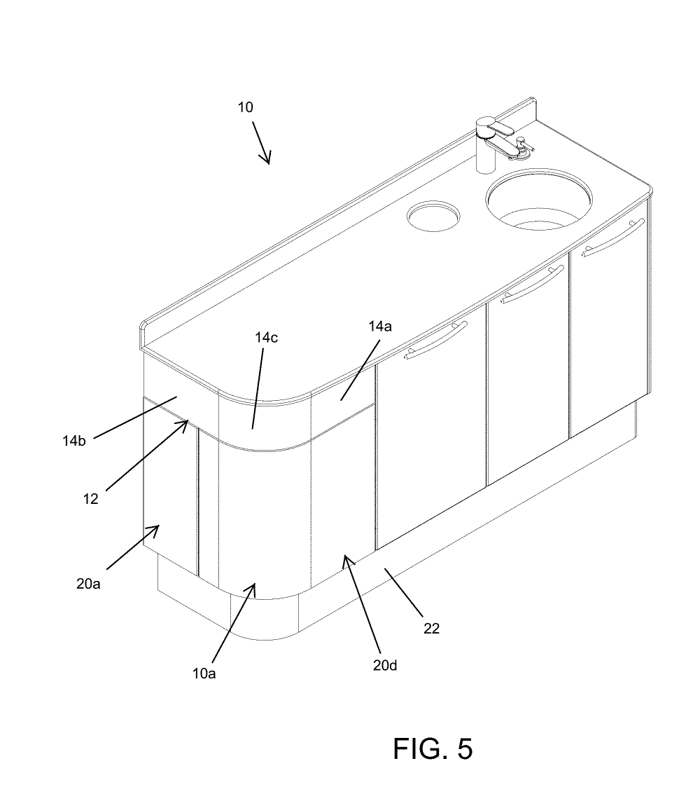

In the illustrated embodiment, the drawer includes the bottom panel and the face panel and two interior panels that extend upward from the bottom panel to define the drawer storage area. The face wall or panel 14 of the corner drawer 12, as shown in FIGS. 1, 4 and 5, is shaped to generally conform and align with the surrounding surfaces or sections of the adjacent side and front walls 20a, 20d of the cabinet 10 when the drawer 12 is in a closed position. Accordingly, with the drawer 12 in the closed position (FIG. 1), the front portion 14a of the face panel 14 may substantially align with or correspond with or match a front face or surface of the front wall 20d of the medical or dental cabinet 10, as generally provided by the adjacent cabinet exterior panel, which may be a front of cabinet drawer or door. Similarly, with the drawer 12 in the closed position (FIG. 5), a side portion 14b of the face panel 14 may substantially align with or correspond with or match a side surface of the side wall 20a of the medical or dental cabinet 10, such as a side panel or other drawer or door adjacent to or above or below the corner drawer. Further, the face panel 14 of the drawer 12 may include a curved portion 14c (FIGS. 4 and 5) that transitions between the front and side portions 14a, 14b of the face panel 14, such as to conform to the corresponding curved panel or section of the base unit 16 below the drawer 12. Optionally, in place of the curved portion 14a, the face panel 14 may alternatively include a generally squared or sharp-angled corner or beveled or angled corner where the front and side portions meet. Similarly, the lower exterior panel below the corner drawer may, optionally, include a corresponding generally squared or sharp-angled corner. However, it is contemplated that the front panel or structure of the corner drawer may be alternatively shaped or sized, such as to not conform or align with the lower exterior panel or adjacent side and front sections of the cabinet.

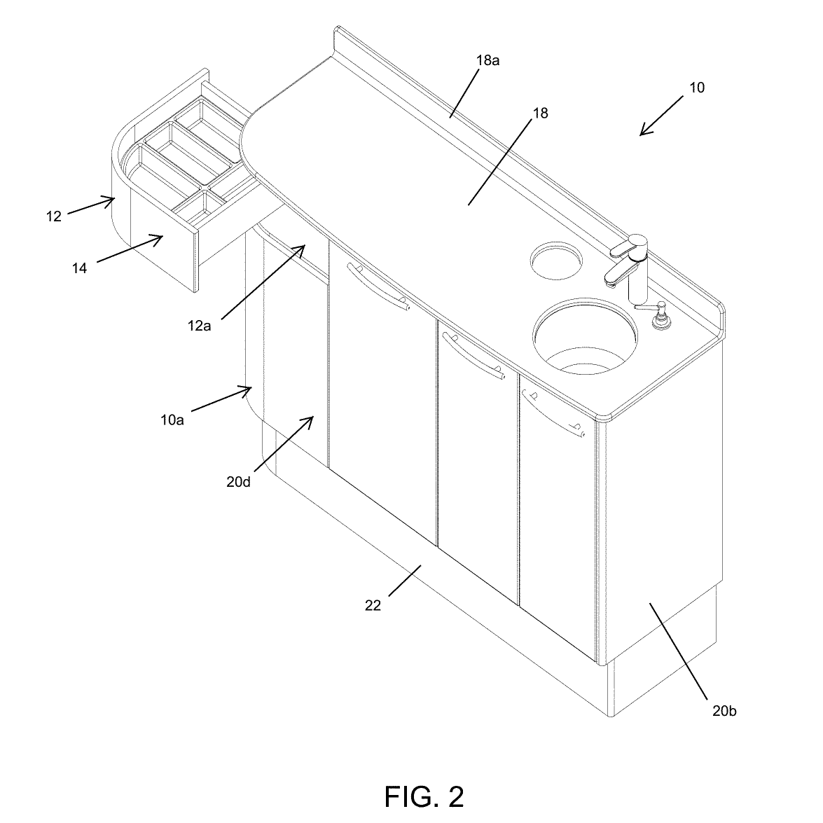

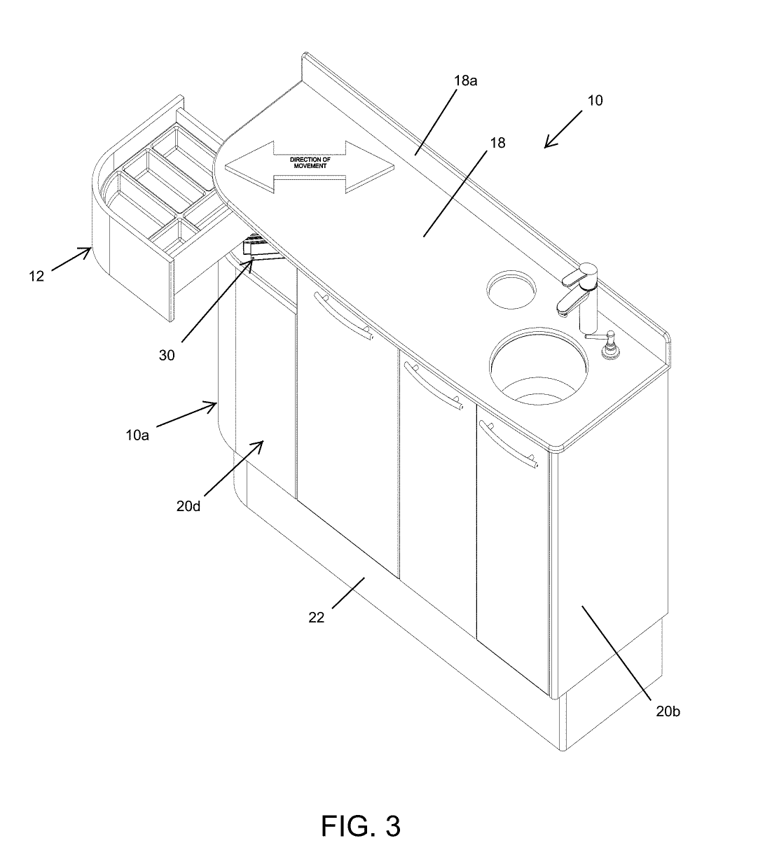

As shown in FIGS. 2-4, 6, and 7, the corner drawer 12 is arranged to travel, such as via a slider or moving mechanism or drawer slide (comprising a track/rail configuration with rollers or bearings that roll to facilitate smooth movement of the drawer relative to the cabinet) 30 (FIG. 3), along a linear path (provided by the track/rail) at least partially into and out of the drawer pocket 12a in the base unit 16 of the cabinet 10, where the drawer 12 moves in a substantially horizontal or level plane that is generally aligned with the ground or upper countertop surface of the cabinet. The moving mechanism 30 may comprise a single slider mechanisms or multiple slider mechanisms, such as the two slider mechanisms 130 shown in the embodiment of the cabinet 110 illustrated in FIG. 12. The slider mechanisms may be conventional drawer slides, such as ball bearing slides, roller slides, guided slides, or other slide configurations that are mounted separately to the drawer or otherwise partially or completely formed integrally between the drawer and cabinet. The corner drawer extends from the cabinet at an angle, such as an oblique angle, that is not parallel or perpendicular with side wall and not parallel or perpendicular with front wall or surface of the medical or dental cabinet, such as preferably an angle generally in the range of twenty to seventy degrees from the side, front, and/or rear wall of the cabinet, or more preferably at an angle in the range of forty to fifty degrees from the side, front, and/or rear wall of the cabinet, such as, for example, at an angle of about forty-five degrees from the side, front, and/or rear wall of the cabinet.

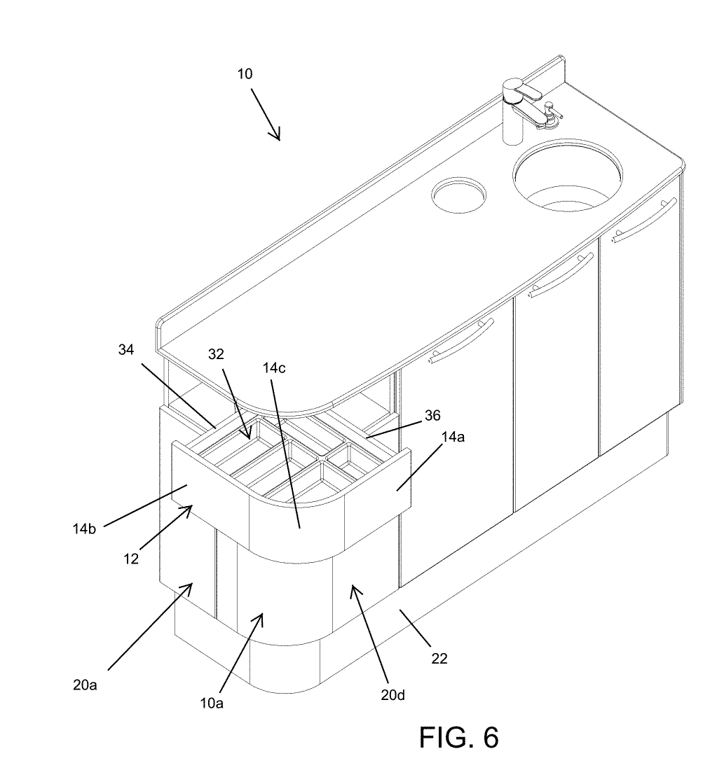

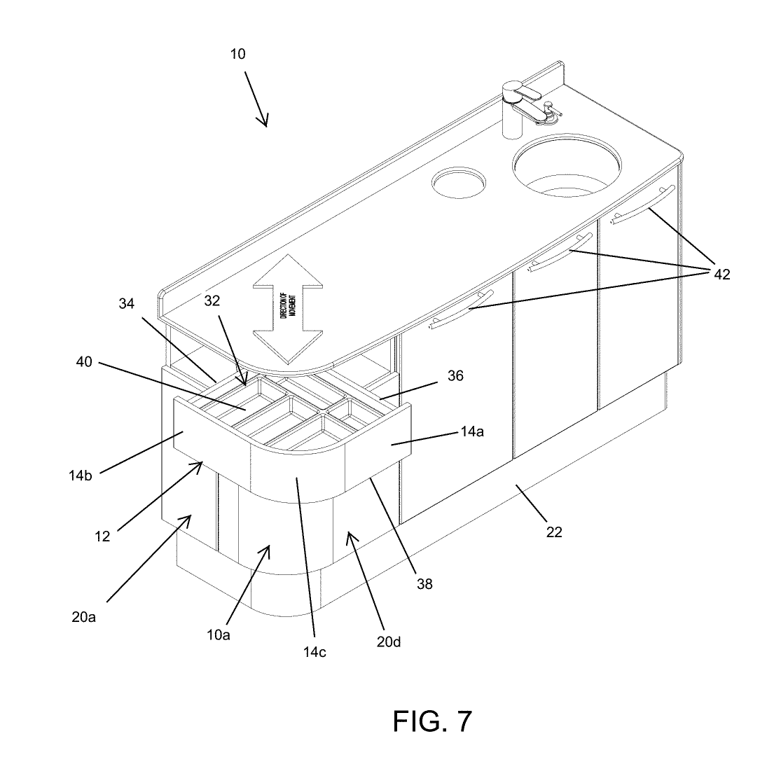

As shown in FIGS. 6-11, the face panel 14 of the corner drawer 12 generally borders at least two sides of the drawer storage area 32. The drawer storage area 32 is further bordered by at least one interior or rear wall or panel, such as a rear wall or panel 34 and a side wall or panel 36, such that the vertical containment walls of the drawer 12 each mate to or attach at a bottom panel 38 of the drawer 12, which generally defines the drawer storage area 32. The drawer storage area 32 may be provided with an organizational insert 40 (FIG. 7) that divides the storage area 32 into separate compartments, such as to accommodate different sized items or devices or the like. The rear panel 34 of the drawer 12, as shown in FIGS. 6-7, is generally parallel with the rear wall 20c of the cabinet and the side panel 36 of the drawer is generally parallel with the side walls 20a, 20b of the cabinet. Further, the bottom panel 38 is relatively planar and is configured to be generally aligned with the countertop or otherwise horizontally arranged relative to the cabinet. However, it is contemplated that the corner drawer may optionally be sized or shaped differently and the vertical containment walls of the drawer each may include different shapes or sizes or configurations.

The drawer 12 may also be provided with a handle or knob or other hardware to facilitate opening and closing the drawer, such as the pulls 42 (FIG. 7) provided at the adjacent drawers or cabinet doors at the front wall 20d of the cabinet 10. Optionally, the drawer may include a push-rebound latch that is disposed or attached between the base unit and the drawer. The push-rebound latch may be configured to hold the drawer in a closed position and, responsive to pushing the drawer inward toward the rear wall of the base unit, release the drawer outward toward an extend position for accessing the storage area. For example, the push-rebound latch may include a spring-loaded magnetic latch or other configuration of such a closure latch.

With reference to FIG. 12, a cabinet 110 includes a corner drawer 112 arranged at a corner portion 110a of the cabinet 110, below the countertop with the face panel of the corner drawer 112 generally shaped to conform and align with the surrounding surfaces or sections of the adjacent side and front walls of the cabinet 110. The face panel of the drawer 112 is curved to conform to the corresponding curved panel or section of the cabinet 110 below the drawer 112. Also similar to the drawer 10 shown in FIGS. 1-11, the vertical containment walls of the drawer 112 each mate to or attach at a bottom panel 138 of the drawer 112, which generally defines the drawer storage area, as shown without the optional organizational insert.

As also shown in FIG. 12, the corner drawer 112 is arranged to travel via two sliders 130 at least partially into and out of the cabinet 110, where the drawer 112 slidably moves in a substantially horizontal or level plane that is generally aligned with the ground or upper countertop surface of the cabinet 110. The corner drawer 112 extends from the cabinet at an oblique angle, such as, for example, about forty-five degrees (or other suitable angle between about 20 degrees and 70 degrees, or between about thirty-five degrees and fifty-five degrees), relative to the side wall of the cabinet 10 and relative to the rear wall of the cabinet 110. In contrast to the extending oblique angle of the slider path of the corner drawer 112, the cabinet 110 shown in FIG. 12 also includes a central drawer that is adjacent to the corner drawer 112 and that extends perpendicularly relative to the rear wall of the cabinet 110 and parallel to the side wall of the cabinet 110.

For purposes of this disclosure, the terms “upper,” “lower,” “right,” “left,” “rear,” “front,” “vertical,” “horizontal,” and derivatives thereof shall relate to the invention as oriented in FIG. 1. However, it is to be understood that the invention may assume various alternative orientations, except where expressly specified to the contrary. It is also to be understood that the specific devices and processes illustrated in the attached drawings, and described in this specification are simply exemplary embodiments of the inventive concepts defined in the appended claims. Hence, specific dimensions and other physical characteristics relating to the embodiments disclosed herein are not to be considered as limiting, unless the claims expressly state otherwise.

Changes and modifications in the specifically described embodiments may be carried out without departing from the principles of the present invention, which is intended to be limited only by the scope of the appended claims as interpreted according to the principles of patent law. The disclosure has been described in an illustrative manner, and it is to be understood that the terminology which has been used is intended to be in the nature of words of description rather than of limitation. Many modifications and variations of the present disclosure are possible in light of the above teachings, and the disclosure may be practiced otherwise than as specifically described.

1. A cabinet for a medical or dental operatory, said cabinet comprising:
a base unit having a side wall and a front wall, wherein the front wall is perpendicular to the side wall;
a support panel movably disposed at the base unit; and
wherein the support panel is disposed at the base unit so as to travel, when moving between an opened position and a closed position relative to the base unit, horizontally along a linear path that is not parallel with the front wall of the base unit and that is not parallel with the side wall of the base unit.

2. The cabinet of claim 1, wherein the support panel includes a front edge and a side edge, and wherein the front edge is aligned with the front wall of the base unit and the side edge is aligned with the side wall of the base unit when the support panel is in the closed position.

3. The cabinet of claim 1, wherein the support panel comprises part of a drawer, and wherein the drawer includes a face panel and at least one interior panel at least partially bordering the support panel and at least partially defining a storage area, wherein the face panel includes a front portion that corresponds with the front wall of the base unit and a side portion that corresponds with the side wall of the base unit.

4. The cabinet of claim 3, wherein the face panel of the drawer comprises a curved portion that transitions between the front portion and the side portion of the face panel.

5. The cabinet of claim 3, wherein the drawer includes a first interior panel parallel with a rear wall of the base unit, and a second interior panel perpendicular to the rear wall of the base unit.

6. The cabinet of claim 1, comprising a moving mechanism disposed between the base unit and a lower surface of the support panel, wherein the support panel moves relative to the base unit via the moving mechanism.

7. The cabinet of claim 1, wherein the linear path along which the support panel travels when opening or closing is at an angle between twenty degrees and seventy degrees relative to the side wall of the base unit.

8. The cabinet of claim 1, wherein the linear path along which the support panel travels when opening or closing is at an angle between forty degrees and fifty degrees relative to the side wall of the base unit.

9. A corner drawer for a medical cabinet, said corner drawer comprising:
a bottom panel that is configured to be horizontally arranged relative to the medical cabinet when the corner drawer is disposed at the medical cabinet;
a face panel attached at the bottom panel to border at least two sides of the drawer storage area;
at least one rear panel attached at the bottom panel;
wherein the face panel and the at least one rear panel circumscribe a drawer storage area at and above the bottom panel;
wherein the face panel includes a front portion configured to align with a front face of the medical cabinet and a side portion configured to align with a side surface of the medical cabinet;
wherein the corner drawer is configured to travel at an angle that is not parallel with the front portion or the side portion of the face panel; and
wherein, with the corner drawer disposed at the medical cabinet, the corner drawer travels, when opening or closing, along a linear path relative to the medical cabinet that is not parallel with the front face of the medical cabinet and that is not parallel with the side surface of the medical cabinet.

10. The corner drawer of claim 9, wherein the face panel of the corner drawer comprises a curved portion that transitions between the front portion and the side portion of the face panel.

11. The corner drawer of claim 9, comprising a moving mechanism disposed at the bottom panel of the corner drawer, wherein the moving mechanism is configured to movably mount the corner drawer at the medical cabinet, and wherein the corner drawer, with the corner drawer disposed at the medical cabinet, moves along the linear path relative to the medical cabinet via the moving mechanism.

12. The corner drawer of claim 11, wherein the moving mechanism is disposed at the bottom panel of the corner drawer such that, with the corner drawer disposed at the medical cabinet, the linear path along which the corner drawer travels is at an angle between twenty degrees and seventy degrees relative to the side wall of the medical cabinet.

13. The corner drawer of claim 11, wherein the moving mechanism is disposed at the bottom panel of the corner drawer such that, with the corner drawer disposed at the medical cabinet, the linear path along which the corner drawer travels is at an angle between forty degrees and fifty degrees relative to the side wall of the medical cabinet.

14. A cabinet for a medical operatory, said cabinet comprising:
a base unit having a corner portion that includes a side section and a front section;
a drawer structure comprising a plurality of vertical containment walls that attach at a bottom panel and that extend upward from the bottom panel;
wherein the drawer structure is movably mounted at the corner portion of the base unit; and
wherein the drawer structure, when opened or closed, travels along a linear path that is at an oblique angle relative to the side section of the corner portion of the base unit and relative to the front section of the corner portion of the base unit.

15. The cabinet of claim 14, wherein the plurality of vertical containment walls of the drawer structure includes a face panel, and wherein the face panel has (i) a front portion that corresponds with the front section of the cabinet when the drawer structure is closed, and (ii) a side portion that corresponds with the side section of the corner portion of the cabinet when the drawer structure is closed.

16. The cabinet of claim 15, wherein the face panel of the drawer structure comprises a curved portion that transitions between the front portion of the face panel and the side portion of the face panel.

17. The cabinet of claim 15, wherein the plurality of vertical containment walls of the drawer structure includes at least one interior panel at least partially bordering the bottom panel, and wherein the at least one interior panel is interior the cabinet when the drawer structure is closed.

18. The cabinet of claim 17, wherein the at least one interior panel comprises a first interior panel parallel with a rear wall of the base unit, and a second interior panel perpendicular to the rear wall of the base unit.

19. The cabinet of claim 17, comprising a moving mechanism disposed between the base unit and the drawer structure, wherein the drawer structure, when opening or closing, moves relative to the base unit via the moving mechanism.

20. The cabinet of claim 19, wherein the linear path along which the drawer structure travels when opening or closing is at an angle between twenty degrees and seventy degrees relative to the side section of the base unit.

21. The cabinet of claim 19, wherein the linear path along which the drawer structure travels when opening or closing is at an angle between forty degrees and fifty degrees relative to the side section of the base unit.
